(19) United States
(12) Patent Application Publication
(10) Pub. No.: US 2016/0296129 A1
(43) Pub. Date: Oct. 13, 2016

(54) BIOLOGICAL INFORMATION DETECTION APPARATUS AND ELECTRONIC APPARATUS

(71) Applicant: Seiko Epson Corporation, Tokyo (JP)

(72) Inventor: Akira Uematsu, Suwa-shi (JP)

(21) Appl. No.: 15/094,275

(22) Filed: Apr. 8, 2016

(30) Foreign Application Priority Data
Apr. 10, 2015 (JP) 2015-080630

(51) Int. Cl. A61B 5/024

(52) U.S. Cl. CPC A61B 5/02427; A61B 5/02438; A61B 2562/0238; A61B 2562/185

(57) ABSTRACT

A biological information detection apparatus includes a sensor having a light emitter that radiates light to a subject and a light receiver that receives light from the subject, and a contact part to be in contact with the subject. Supposing that light power from the light emitter passing through a living body and entering the light receiver is PS and a distance from the light receiver to an end portion of the contact part is rN, and light power of disturbance light from outside of the end portion passing through the living body and entering the light receiver is PN(rN) as a function of rN, an attenuation rate α of the disturbance light is set to satisfy rN≦10 mm and α≦PS/{PN(rN)×1000}.

### CROSS-REFERENCES TO RELATED APPLICATIONS

This application claims priority to Japanese Patent Application No. 2015-080630, filed Apr. 10, 2015, the entirety of which is herein incorporated by reference.

### BACKGROUND

1. Technical Field

The present invention relates to a biological information detection apparatus, an electronic apparatus, etc.

2. Related Art

In related art, a technique of detecting biological information of a subject (user) using a photoelectric sensor is known. The biological information here is e.g. pulse wave information. A signal representing pulsation of a vessel under the skin is acquired by a light receiver receiving reflected light in the vessel or transmitted light transmitted through the vessel of light radiated from a light emitter.

Patent Document 1 (JP-A-2014-54447) discloses a pulse wave measuring apparatus using a photoelectric sensor. The technique disclosed in Patent Document 1 is based on consideration of accurate measurement of a pulse rate even when the user is not at rest by changing a peak search range of a frequency according to exercise intensity.

As disclosed in Patent Document 1, when the biological information is detected using the photoelectric sensor, the relationship between a signal component and a noise component should be considered for appropriate detection. The signal component here corresponds to light radiated from the light emitter, passing through the living body, and entering the light receiver. On the other hand, the noise component corresponds to disturbance light. Various lights are considered as the disturbance light, and the light having the most impact is sunlight.

In this regard, downsizing of the biological information detection apparatus leads to increase of the intensity of sunlight (light power) passing through the living body and entering the light receiver. There are detailed description regarding propagation of light within the living body in Patent Document 2 (JP-A-7-148170) and Non-patent Document (A N Bashkatov, E A Genina, V I Kochubey and V V Tuchin, Optical properties of human skin, subcutaneous and mucous tissues in the wavelength).

The biological information detection apparatus is intended to be realized as a wearable apparatus worn by a user and downsizing is required, however, there is a relationship such that, as the apparatus is made smaller, the noise influence is stronger. In related art, to improve the detection accuracy of the biological information detection apparatus, various techniques of increasing the light intensity of the light emitter, increasing the sensitivity of the light receiver, and using an optical filter have been widely used. However, there is no disclosure of technologies in view of suppression of the influence of disturbance light (sunlight), particularly, in view of appropriate settings of parameters of the size of the apparatus and the optical attenuation rate (stop rate) for downsizing of the biological information detection apparatus.

### SUMMARY

An advantage of some aspects of the invention is to provide a biological information detection apparatus, an electric apparatus, etc. that compatibly realize both downsizing of the apparatus and suppression of disturbance light influence.

An aspect of the invention relates to a biological information detection apparatus including a sensor having a light emitter that radiates light to a subject and a light receiver that receives light from the subject, and a contact part to be in contact with the subject, wherein, supposing that light power from the light emitter passing through a living body as the subject and entering the light receiver is PS and a distance from the light receiver to an end portion of the contact part is rN, and light power of disturbance light from outside of the end portion passing through the living body and entering the light receiver is PN(rN) as a function of rN, an attenuation rate α of the disturbance light is set to satisfy rN≦10 mm and α≦PS/{PN(rN)×1000}.

In the aspect of the invention, the distance rN to the end portion of the contact part is suppressed to be smaller and the attenuation rate α is set from PN as the function of rN representing the light power of disturbance light and PS representing light power of signal light. In this manner, while the biological information detection apparatus is downsized, the influence of disturbance light can be suppressed and appropriate detection can be performed.

In the aspect of the invention, the attenuation rate α may be set to satisfy rN≦10 mm and α≦1/30.

With this configuration, specific values can be set as rN and α.

In the aspect of the invention, the attenuation rate α of the disturbance light may be set so that, as rN is smaller in the range of rN≦10 mm, α may be smaller in the range of α≦1/30.

With this configuration, the biological information detection apparatus can be further downsized by further increasing the degree of attenuation (decreasing α).

In the aspect of the invention, the attenuation rate α may be set to satisfy rN≦8 mm and α≦1/100.

With this configuration, specific values may be set as rN and α.

In the aspect of the invention, the attenuation rate α may be set to satisfy rN≦5 mm and α≦1/1000.

With this configuration, specific values can be set as rN and α.

In the aspect of the invention, an optical filter provided in the light receiver and limiting light entering the light receiver may be further provided, and the attenuation rate α may be set by the optical filter.

With this configuration, the attenuation rate α can be realized by the optical filter.

In the aspect of the invention, the optical filter may include a multilayer filter.

With this configuration, the attenuation rate α can be realized by the optical filter at least including the multilayer filter.

In the aspect of the invention, a casing in which the sensor is provided may be provided, and the contact part may be a casing surface on which the casing is in contact with the subject when the casing is attached to the subject.

With this configuration, the biological information detection apparatus having the casing can be realized and the contact part can be realized by the casing surface.

In the aspect of the invention, a casing in which the sensor is provided may be provided, and the contact part may be a surface of the casing facing the subject when the casing is attached to the subject.

With this configuration, the biological information detection apparatus having the casing can be realized and the contact part can be realized by the surface of the casing facing the subject.

In the aspect of the invention, a casing in which the sensor is provided may be provided, and the contact part may be a projecting region projecting toward the subject of a surface of the casing facing the subject when the casing is attached to the subject.

With this configuration, the biological information detection apparatus having the casing can be realized and the contact part can be realized by the projecting region of the surface of the casing facing the subject.

In the aspect of the invention, a processor that performs detection processing of biological information based on sensor information from the sensor may be further provided.

With this configuration, the biological information can be detected based on the sensor information.

In the aspect of the invention, the biological information may be pulse wave information.

With this configuration, the pulse wave information can be detected based on the sensor information.

In the aspect of the invention, supposing that light power from the light emitter passing through the living body, entering the light receiver, and corresponding to the pulse wave information is PM, PM≧PS/1000 may hold.

With this configuration, the attenuation rate α can be determined in consideration of a relationship between the light power PS of the signal light and the light power PM corresponding to the pulse wave information.

Another aspect of the invention corresponds to an electric apparatus including the information detection apparatus described above.

### BRIEF DESCRIPTION OF THE DRAWINGS

The invention will be described with reference to the accompanying drawings, wherein like numbers reference like elements.

FIGS. 1A and 1B are a plan view and a sectional view showing an arrangement example of a light emitter, a light receiver, and a casing and a relationship between signal light and disturbance light.

FIG. 2 shows relationship examples among a wavelength, an absorption coefficient, and a scattering coefficient.

FIG. 3 shows relationship examples between a propagation distance within a living body and light power.

FIG. 4 shows spectral characteristics of light power of sunlight radiated to a skin.

FIG. 5 shows relationship examples between a wavelength band of sunlight and light power.

FIG. 6 shows an example of a calculation technique of light power of disturbance light passing through the living body and entering the light receiver.

FIG. 7 shows a relationship example between a distance to an end of a contact part and disturbance light power.

FIG. 8 shows a setting example of an attenuation rate.

FIG. 9 is a sectional view of the light receiver and a multilayer filter according to the embodiment.

FIG. 10 is a sectional view when an angle-limiting filter is provided in the light receiver.

FIGS. 11A and 11B are explanatory diagrams of a forming process of an angle-limiting filter.

FIG. 12 is an explanatory diagram of the forming process of the angle-limiting filter.

FIG. 13 shows a configuration example of the biological information detection apparatus.

FIGS. 14A and 14B are appearance diagrams of the biological information detection apparatus.

FIG. 15 is another appearance diagram of the biological information detection apparatus.

FIGS. 16A to 16C show specific examples of the contact part.

### DESCRIPTION OF EXEMPLARY EMBODIMENTS

As below, an embodiment will be explained. Note that the embodiment to be explained does not unduly limit the subject matter of the invention described in the appended claims. Further, not all of the configurations explained in the embodiment are necessarily the essential component elements of the invention.

### 1. Technique of Embodiment

First, a technique of the embodiment will be explained. As described above, the techniques using photoelectric sensors for detection of biological information are widely known. The photoelectric sensor includes a light emitter and a light receiver and detects biological information by receiving light radiated from the light emitter and passing through a living body with the light receiver. In this regard, the light receiver may be an element that can detect light (for example, perform photoelectric conversion), not for distinction of the kind of light entering the light receiver. For example, the light receiver does not distinguish a light source of the entering light or a propagation path of the light. Accordingly, when disturbance light enters, it is impossible for the light receiver to distinguish the disturbance light from light radiated from the light emitter and passing through the living body.

It is desired to use the light radiated from the light emitter and passing through the living body for detection of the biological information (hereinafter, also referred to as “signal light” as light corresponding to a signal component), and the disturbance light is regarded as a noise component. The light having the highest contribution in the disturbance light is sunlight and the sunlight will be explained as below.

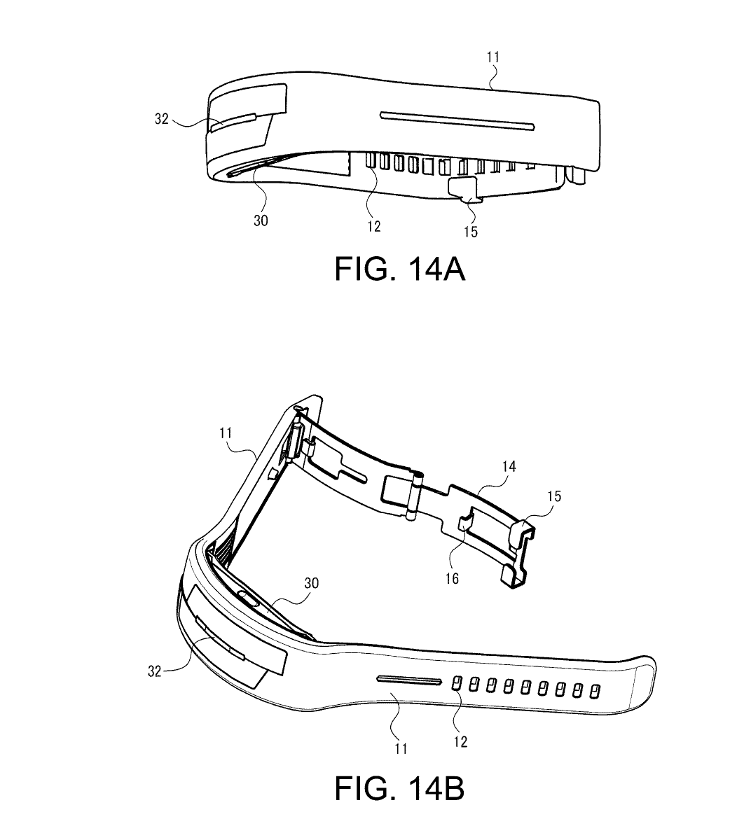

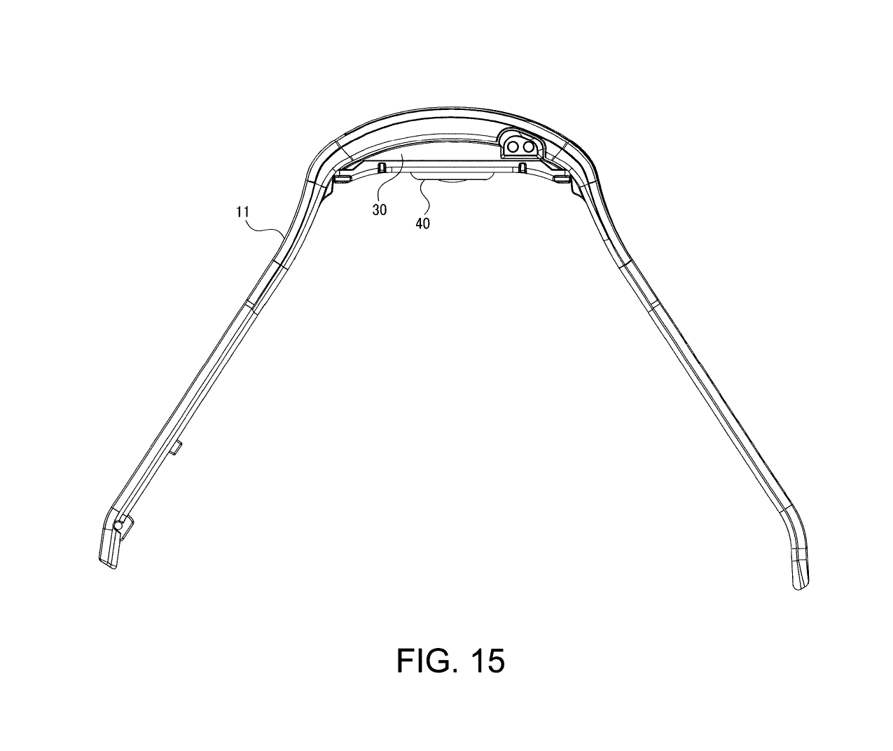

Accordingly, in the biological information detection apparatus of related art, a structure that prevents entrance of sunlight in the light receiver is usually used. For example, in a wristwatch-type (band-type) biological information detection apparatus, which will be described later using FIG. 14A to 15 and etc., a photoelectric sensor (sensor 40) is provided on a contact surface side of a main body part (case part 30, casing) to be in contact with a subject. According to the configuration, in a state of detection of the biological information, i.e., in a state in which the wristwatch-type apparatus is attached to an arm of a user, the light receiver is sandwiched between the casing containing the photoelectric sensor (and members forming the biological information detection apparatus including a circuit board) and a living body. That is, the casing serves as a light-blocking member and direct entrance of sunlight into the light receiver may be suppressed.

However, as known from the light radiated from the light emitter and passing through the living body, in a narrow sense, reaching a vessel region, reflected by the vessel, and reaching the light receiver in the detection of the biological information, the light has a property of propagating within the living body. That is, the direct entrance of sunlight into the light receiver may be suppressed using the casing or the like as a light-blocking member, however, the light entering the living body from the position not blocked by the light-blocking member, propagating within the living body, and entering the light receiver is harder to be suppressed.

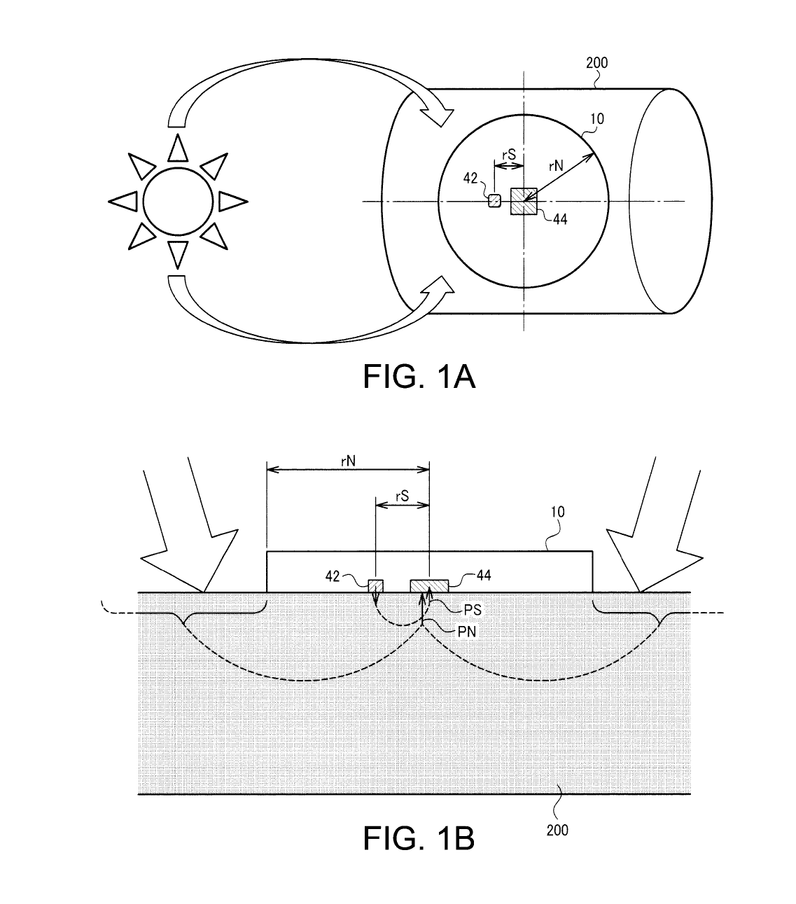

A specific example is explained using FIGS. 1A and 1B. FIG. 1A is a plan view showing an arrangement example of a light emitter, a light receiver, and a casing and a relationship between signal light and disturbance light, and FIG. 1B is a sectional view thereof. As shown in FIG. 1A, here, an example in which a circular light-blocking area is provided, specifically, an example using a casing in a circular plate shape is assumed. Further, a light emitter 42 and a light receiver 44 are provided in positions between the casing and a living body 200. Note that, in FIG. 1A, the light receiver 44 is provided at the center of the casing and the light emitter 42 is provided in the position shifted from the center, however, the position relationship is not limited to that. An arrangement in which a midpoint of the light emitter 42 and the light receiver 44 is at the center of the casing, an arrangement in which the light emitter 42 is at the center of the casing may be employed.

In this case, as shown in FIG. 1A, sunlight enters another area than the light-blocking area of a living body surface (skin). Accordingly, as shown in FIG. 1B, light LS radiated from the light emitter 42 and passing through the living body and sunlight LN entering from a point outside of the light-blocking area and passing through the living body enter the light receiver 44.

Here, supposing that light power of LS is PS and light power of LN is PN, of output values of the light receiver 44, a signal component corresponds to PS and a noise component corresponds (in a narrow sense, is proportional) to PN, and an S/N ratio takes a value corresponding to PS/PN.

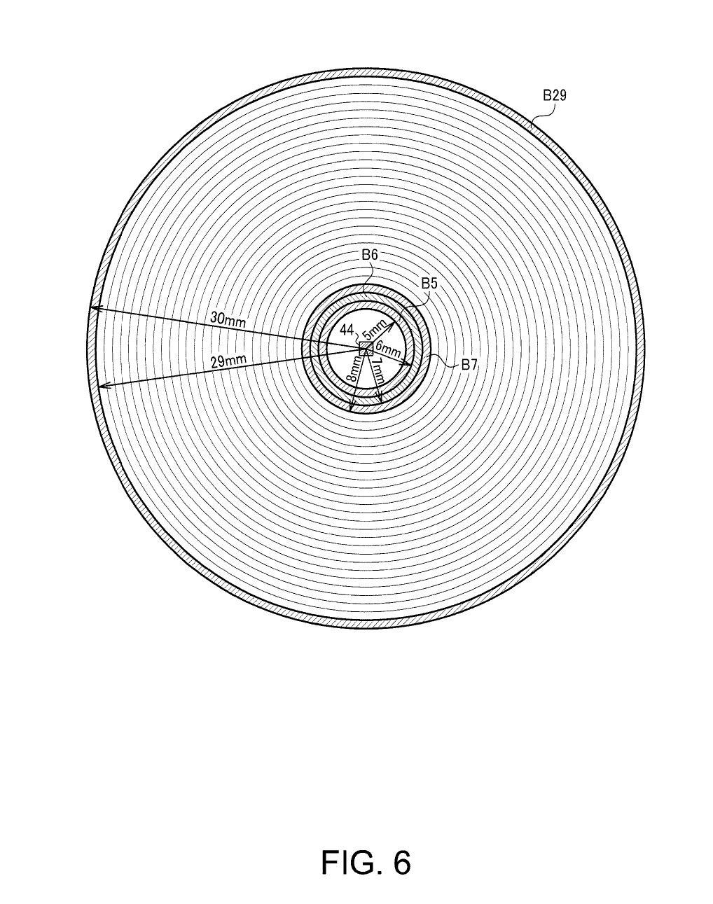

Further, supposing that given one point outside of the light-blocking area is k and light power of sunlight entering from k and passing through the living body is pn(k), PN takes a value obtained by integration of pn(k) over the entire region outside of the light-blocking area. For example, as shown in FIG. 1B, PN is a sum of light power of sunlight entering from a range in which the distance from the light receiver is equal to or more than rN, passing through the living body, and entering the light receiver 44. Note that a specific calculation example will be described later using FIG. 6.

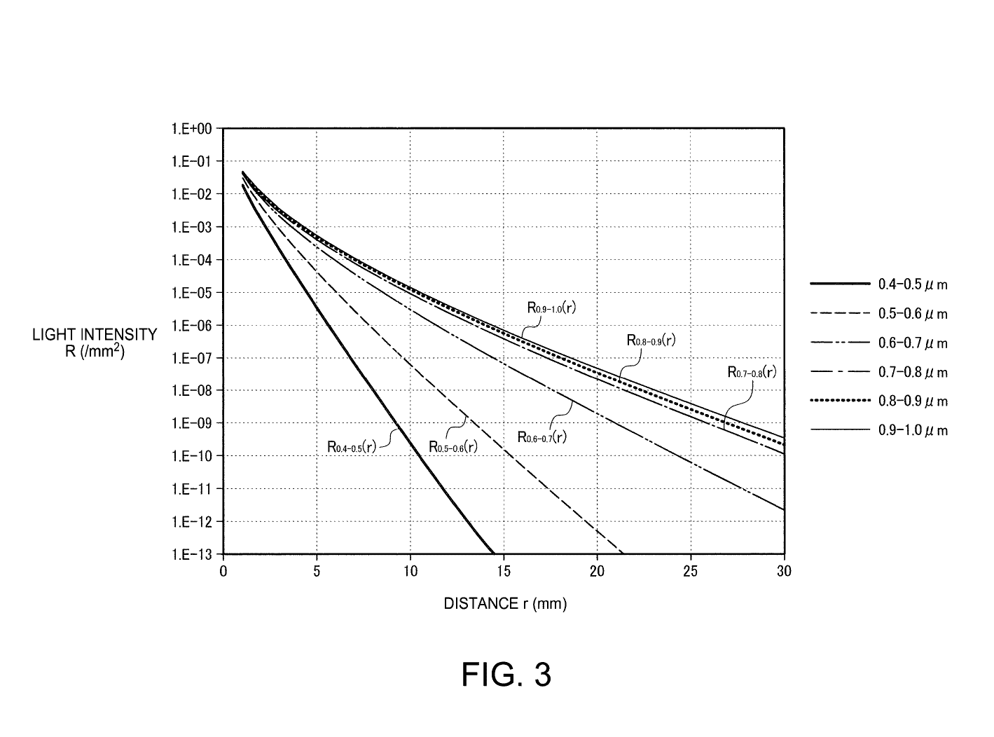

That is, the noise component PN can be made smaller by making the region outside of the light-blocking area smaller, in other words, making the light-blocking area larger. Particularly, as will be described later using FIG. 3, as the distance of propagation within the living body in the planar direction is longer, attenuation of light within the living body is exponentially larger. Namely, the effect of reduction of PN by increasing the light-blocking area is greater in view of suppression of entrance of sunlight from a position relatively near the light receiver 44.

Here, in view of the fact that the size of the light-blocking area corresponds to the size of the casing or the similar member of the biological information detection apparatus, as the size of the biological information detection apparatus is made larger, the influence of noise due to sunlight can be further suppressed. Conversely, as the biological information detection apparatus is made smaller, the influence of noise due to sunlight is larger.

Accordingly, the biological information detection apparatus of related art is not extremely downsized. Specifically, in a wristwatch-type apparatus, a diameter of a main body part (a casing part except a band part) is larger than 20 mm, and, for rN in FIG. 1B, rN>10 mm. However, the size exceeding 20 mm in diameter corresponds to a large-sized wristwatch and a large size in comparison with a thickness (diameter) of a human wrist. That is, it is possible that the biological information detection apparatus having the size is an obstacle when worn, and the apparatus is particularly unsuitable for women and children having thinner wrists. Note that, here, the distance rN is a distance from the center of the light receiver 44 in the plan view as shown in FIG. 1A. Or, when the light receiver has a rectangular shape in the plan view, an intersection of diagonal lines may be defined as the center of the light receiver and the distance rN may be a distance from the intersection of the diagonal lines. Further, the position of the light receiver 44 is not limited to a position with reference to the center, but various representative positions may be used. The representative position may be a position of the center of gravity, a position corresponding to an end point, or another position contained in the light receiver 44 in the plan view. In this case, the distance rN is a distance from the representative position of the light receiver 44.

Accordingly, the applicant proposes a technique of suppressing the noise component (PN) to the level at which the biological information can be detected while realizing downsizing such that rN may be 10 mm or less. Specifically, a biological information detection apparatus 10 according to the embodiment includes a sensor 40 having the light emitter 42 that radiates light to a subject and the light receiver 44 that receives light from the subject, and a contact part 50 to be in contact with the subject. Supposing that light power from the light emitter 42 passing through the living body and entering the light receiver is PS and a distance from the light receiver 44 to an end portion of the contact part 50 is rN, when light power of disturbance light from outside of the end portion passing through the living body and entering the light receiver 44 is PN (rN) as a function of rN, an attenuation rate α of disturbance light is set to satisfy the following expression (1) and expression (2).

rN≦10 mm (1)

α≦PS/{PN(rN)×1000} (2)

Here, the contact part 50 refers to a part of the biological information detection apparatus 10 in contact with the living body when biological information is detected, and, in the case of a wristwatch-type biological information detection apparatus 10 to be described later using FIGS. 14A to 15, refers to a surface on the living body side of the case part 30 (casing). Specifically, if the casing of the biological information detection apparatus 10 is realized by a combination of the main body part and a back cover part, the back cover part may be the contact part 50, or, if the casing is integrally formed by extrusion molding or the like, a part on the living body side of the casing may be the contact part. Further, the contact part 50 is not necessarily a whole member provided on the living body side of the biological information detection apparatus 10. For example, in the case where the biological information detection apparatus 10 has a back cover part, a part of the back cover part may come into contact with the living body and the other parts not (float with respect to the living body). The contact part 50 in this case refers to a part in contact with the living body of the back cover part.

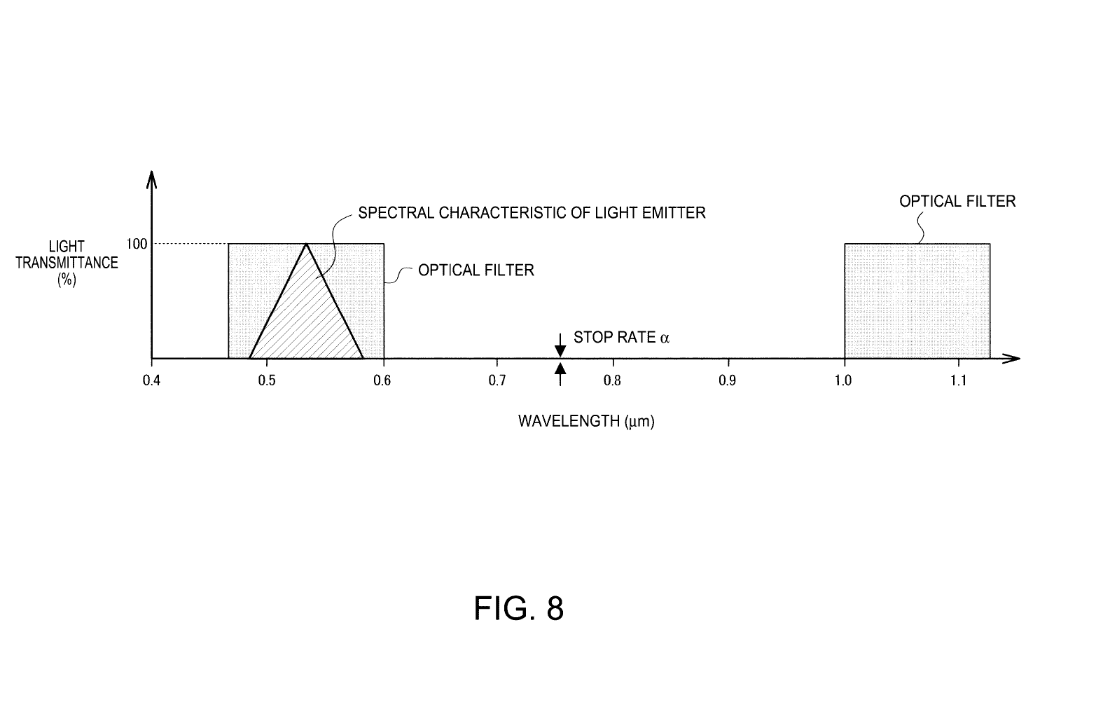

The attenuation rate α of disturbance light is a positive number that satisfies α<1, and, when disturbance light having light power PN enters the light receiver 44, represents a property that attenuates the light power to α×PN. Note that, as will be described later using FIG. 8, it is not preferable to attenuate signal light, and actually, light in a wavelength band corresponding to the signal light may not be attenuated (transmitted at 100% or a rate close thereto), but light power in the other wavelength bands may be attenuated to α times.

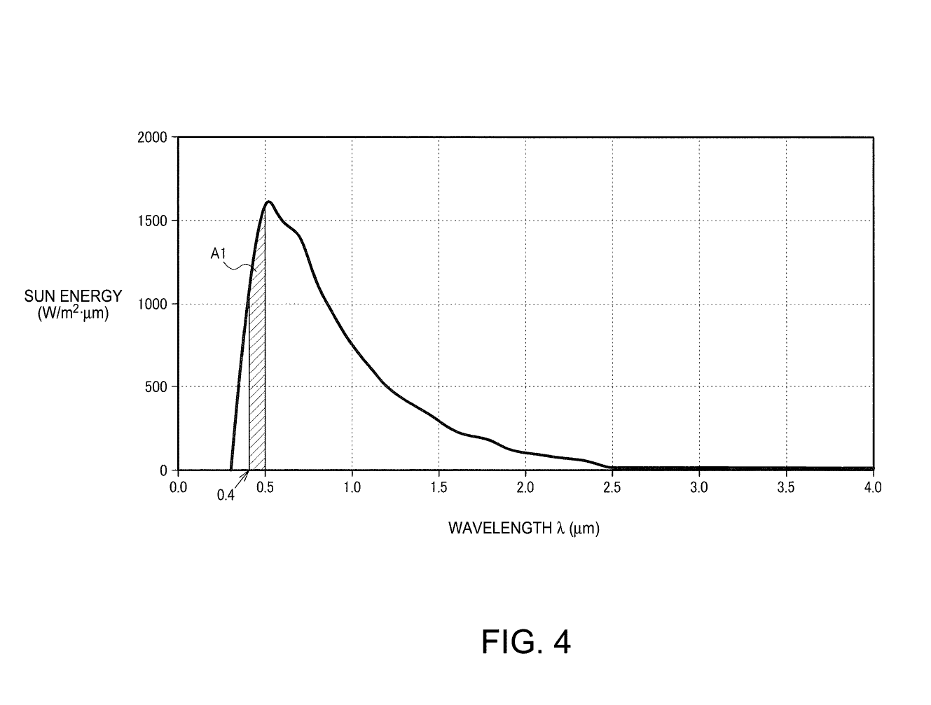

In this case, regarding the disturbance light, the light in the wavelength band corresponding to the signal light is not attenuated, and the attenuation of light power may not strictly be α×PN. However, as will be described later using FIG. 3, the degree of attenuation of the light propagating within the living body is larger as the wavelength is shorter. Accordingly, of spectral characteristics of sunlight (to be described later using FIG. 4), the degrees of attenuation of lights of 0.4 μm to 0.6 μm corresponding to the shorter wavelength bands are larger, and light power of the disturbance light reaching the light receiver 44 is negligibly small. As the signal light used here, e.g. light from 0.5 μm to 0.6 μm corresponding to the wavelength band of green or the like is assumed. Accordingly, of disturbance light, even if light in the wavelength band corresponding to the signal light is transmitted (not attenuated at α), consideration of the resulting influence on the light power PN is not required. Namely, in the embodiment, an optical filter or the like having the property as shown in FIG. 8 is used, and thereby, the light power of disturbance light may be considered to be α×PN.

That is, the disturbance light-induced light power finally received by the light receiver 44 is PN (rN)λα. By setting α to satisfy the expression (2), PN (rN)λα≦PS/1000, i.e. the attenuation rate for suppressing the disturbance light-induced light power to 1/1000 of the light power of the signal light can be set.

The pulse wave information is obtained from fluctuations in blood flow, and thus, a signal actually representing the pulse wave information is an AC component of the signal light LS (i.e., an AC component as an amount of fluctuation of PS). Further, Patent Document 1 describes that, with respect to a signal corresponding to PS, a signal value smaller by about two orders of magnitude corresponds to the AC component, and it is known from experiments by the applicant that the AC component is about 1/800 to 1/300 of a DC component. Namely, the signal component corresponding to the pulse wave takes a value from about PS/800 to PS/300, and thus, if the signal due to disturbance light is smaller to the level that can be sufficiently distinguished from the signal component corresponding to the pulse wave, appropriate pulse wave information (in a broad sense, biological information) can be detected. In this regard, if the expression (2) is satisfied, PN(rN)×α≦PS/1000, and the noise component can be made sufficiently smaller with respect to the component corresponding to pulse wave.

Furthermore, these settings are made under the condition of the expression (1), and thereby, downsizing of the biological information detection apparatus can be realized. Namely, according to the technique of the embodiment, both downsizing of the biological information detection apparatus and suppression of influence by disturbance light can be compatibly realized.

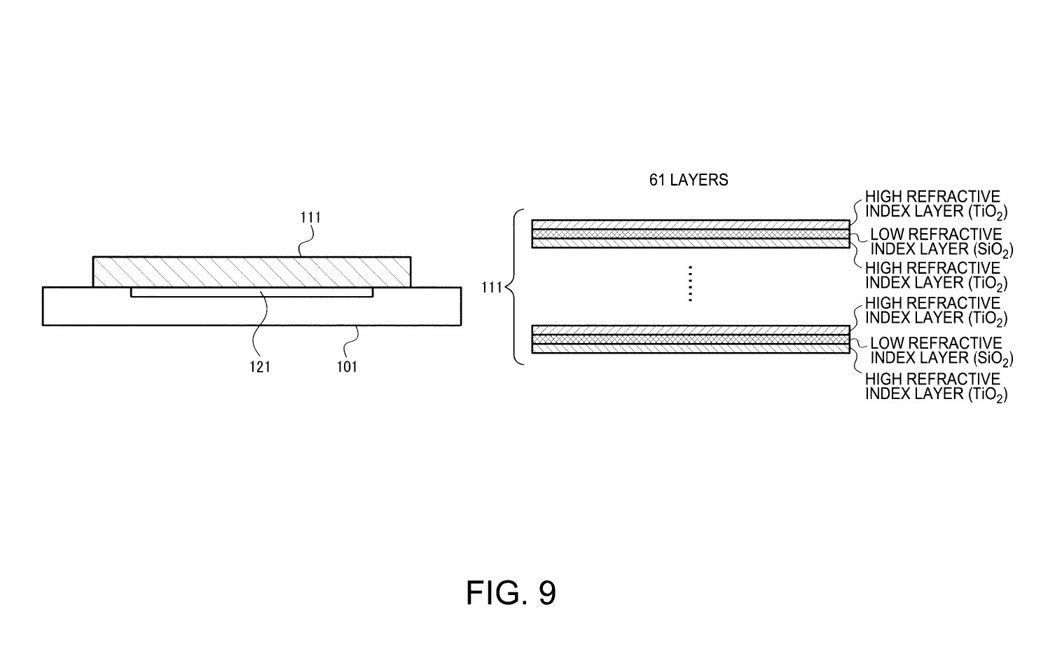

Note that, through specific numerical values will be described later, to satisfy both the expressions (1) and (2), α takes a very small value equal to or less than 1/30. That is, it is necessary to make the degree of attenuation of light very large and, as an example, a high-performance optical filter should be realized. In this regard, the applicant uses a multilayer filter, an angle-limiting filter, which will be described later using FIGS. 9 to 12, or the like, and the condition on α can be satisfied.

As below, a method of obtaining a specific numerical value of α that satisfies the expressions (1) and (2) based on a propagation simulation of light within the living body will be explained. In addition, a specific example of α in further downsizing (when rN is set to be smaller than 100 mm) will be explained. Further, the optical filter will be explained as a specific configuration example that realizes α, and finally, a specific configuration example of the biological information detection apparatus 10 according to the embodiment will be explained.

### 2. Setting Examples of Distance rN Between Light Receiver and End Portion and Attenuation Rate α

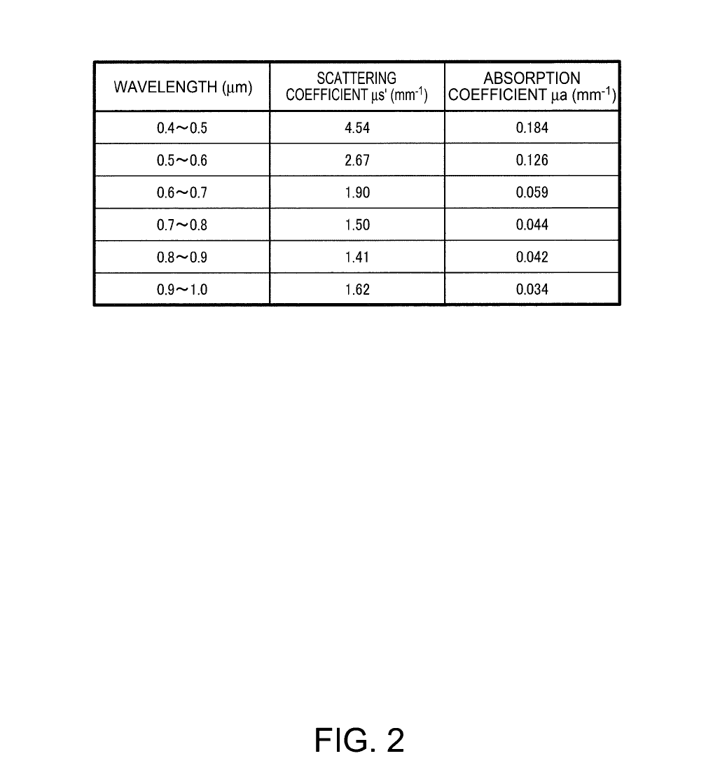

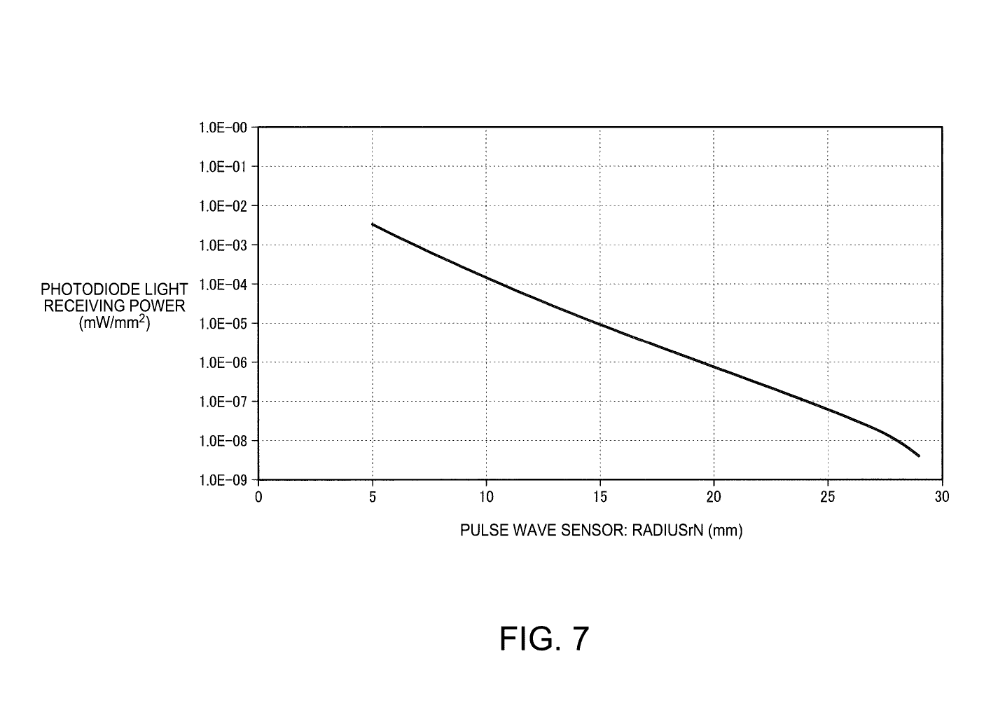

Specific setting examples of rN and α will be explained. First, principles of propagation of light within the living body etc. of the embodiment will be explained using FIGS. 2 and 7. Then, the optical filter as a specific configuration example for realizing the attenuation rate α will be explained.

### 2.1 Principles of Technique of Embodiment

Various propagation characteristics of light within a living body, particularly, within a skin are known. For example, Patent Document 2 discloses the following expression (3). In the following expression (3), R(r) is light intensity of propagation at a distance of r mm, Z₀ is a virtual incident depth, D is a diffusion coefficient, and μa is an absorption coefficient. Further, the diffusion coefficient D is expressed by the following expression (4), and μs′ in the expression (4) is a scattering coefficient (correction scattering coefficient).

$$\begin{matrix}R ( r ) = \frac{{Z}_{0}}{2 π} {( {r}^{2} + {Z}_{0} )}^{- 3 / 2} \times ( 1 + \sqrt{\frac{μ a}{D}} \sqrt{{r}^{2} + {Z}_{0}^{2}} ) \times exp ( - \sqrt{\frac{μ a}{D}} \sqrt{{r}^{2} + {Z}_{0}^{2}} ) & ( 3 ) \\ D = \frac{1}{3 ( μ a + μ {s}^{′} )} & ( 4 )\end{matrix}$$

Further, there is a disclosure of the absorption coefficient μa and the scattering coefficient μs′ in FIG. 2. (a) and (b) of Non-patent Document 1, and relationships between wavelength bands of light and μa, μs′ in the wavelength bands may be derived.

FIG. 3 may be derived using the above expressions (3), (4) and FIG. 2. FIG. 3 shows relationships between the propagation distance r within the skin and light power (light intensity) R. Specifically, for the respective wavelength bands, the values of μa, μs′ shown in FIG. 2 may be substituted into the above expressions (3), (4). Note that, for deriving FIG. 3, as a virtual incident depth, Z₀=1 mm is used. Further, in FIG. 3, light intensity at distance r=0 is “1”. Namely, FIG. 3 is a graph showing the degree of attenuation of light power for propagation by the distance r.

As is known from FIG. 3, the shorter the wavelength, the larger the degree of attenuation of light power with respect to the distance, and the longer the wavelength, the smaller the degree of attenuation. The degree of attenuation of red light in a relatively long wavelength band (e.g. 0.6 μm or longer) is smaller, and, when signal light in the wavelength band is used, the influence of disturbance light is larger. This is because disturbance light entering a position farther from the light receiver 44 remaining at the smaller degree of attenuation within the skin also reaches the light receiver 44. In the embodiment, the explanation is made using green light (in a wavelength band e.g. from 0.5 μm to 0.6 μm) in consideration of that. Note that an embodiment using a wavelength band of red or the like is not denied.

Here, when light from 0.5 μm to 0.6 μm is used as the signal light and a distance rS between the light emitter 42 and the light receiver 44 is 2 mm, a value of R=0.00471 (/mm²) is obtained as light power from FIG. 3. That is, the light power of the light radiated from the light emitter 42 is attenuated to 0.00471 times and received by the light receiver 44. Supposing that light power of light radiated from the light emitter 42 and received by the light receiver 44 not through the living body is 1 mW, light power PS of the signal light with the setting of rS=2 mm is expressed by the following expression (5).

PS=1 mW×0.00471/mm²=4.71×10⁻³ mW/mm² (5)

By various techniques of increasing the light emission intensity of the light emitter 42, increasing the directivity of light by providing a lens or the like in the light emitter 42, or increasing light sensitivity of the light receiver 44, light power of light radiated from the light emitter 42 and received by the light receiver 44 not through the living body may be increased and PS can be increased. Further, PS may be increased by decreasing the distance rS between the light emitter 42 and the light receiver 44. Note that, under present circumstances, various products having different performance, arrangements, etc. of the light emitter 42 and the light receiver 44 are known, however, many of those different products are at levels of light power PS of signal light of the above described expression (5). Therefore, in the following explanation of the embodiment, settings of parameters etc. are made with PS as the value expressed by the expression (5). Here, the distance rS is a distance from the center of the light emitter 42 and the center of the light receiver 44 in the plan view as shown in FIG. 1A or in the sectional view as shown in FIG. 2B. Further, when the light emitter and the light receiver have rectangular shapes in the plan view, the distance may be a distance between intersections of diagonal lines. Note that the reference of the position of the light receiver 44 may be set to a representative position as is the case of the distance rN. Further, the reference of the position of the light emitter 42 may be set to a representative position, not the center. Namely, the distance rS may be a distance from a representative position of the light emitter to a representative position of the light receiver.

As described above, the AC component corresponding to pulse wave of PS is about 1/800 to 1/300. In other words, supposing that light power from the light emitter 42 passing through the living body and entering the light receiver 44, and corresponding to pulse information is PM, PM≧PS/1000. PM here corresponds to e.g. power of the AC component of PS.

In consideration of that, it is preferable to suppress light power of disturbance light received by the light receiver 44 to about 1/1000 of PS. That is, the light power of disturbance light may be set to 4.71×10⁻⁶ mW/mm² as 1/1000 of the above expression (5) or less. In this manner, processing may be performed in consideration of the light power corresponding to the pulse wave information. Accordingly, the magnitude of the light power PM corresponding to the pulse wave information with respect to the light power PN corresponding to noise can be appropriately set (specifically, PM>PN).

Thus, next, an evaluation of the light power PN of disturbance light (sunlight) entering the light receiver 44 is made. First, FIG. 4 shows spectral characteristics of light power of sunlight. The horizontal axis indicates wavelength and the vertical axis indicates to light power per unit wavelength (the unit is W/m²·μm). FIG. 4 shows light power of sunlight falling down to the ground surface and may be considered to show light power of sunlight radiated to the living body (the user skin).

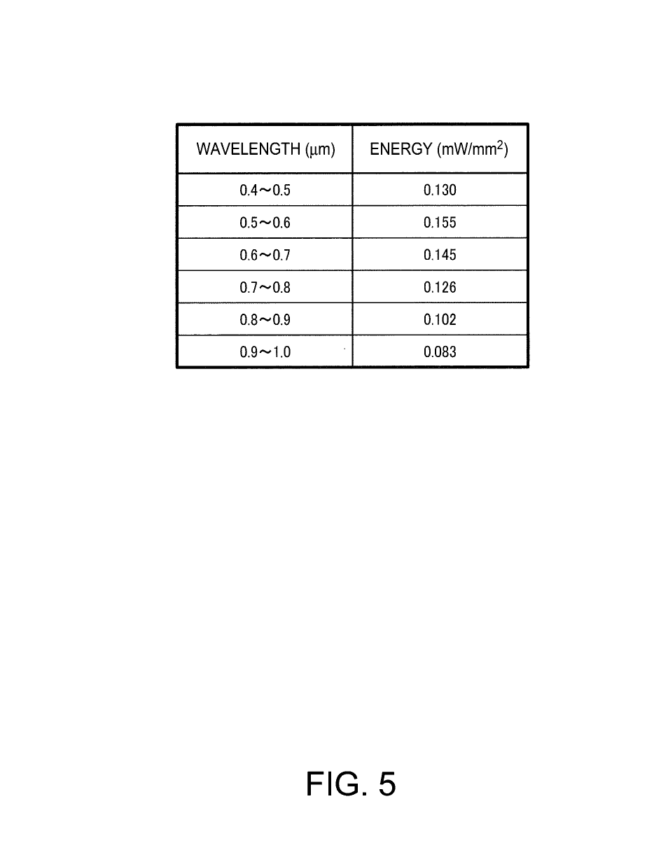

FIG. 5 is formed by obtaining light power of sunlight with respect to each wavelength band from FIG. 4. In FIG. 5, wavelength bands formed by sectioning a range from 0.4 μm to 1.0 μm at intervals of 0.1 μm is considered. For example, in FIG. 4, information corresponding to an area of a range shown by A1 is light power per unit area in the wavelength band from 0.4 μm to 0.5 μm. Note that W and m are used for the unit in FIG. 4 and mW and mm are used in FIG. 5, and thus, conversion between them should be performed.

As will be described later using FIG. 9 etc., setting of the sensitivity wavelength range of the light receiver 44 (photodetection region 121) to a range e.g. from 0.3 μm to 1.1 μm or from 0.4 μm to 1.0 μm is considered. In this case, even when the light having another wavelength than that in the sensitivity wavelength range enters, the light is not detected by the light receiver 44 and the light does not contribute as a signal or noise. Therefore, in the embodiment, for the evaluation of PN, processing is performed on a wavelength band assumed as the sensitivity wavelength range of the light receiver 44, specifically, the range from 0.4 μm to 1.0 μm as shown in FIG. 5 as the wavelength band. Note that the wavelength band may be set to from 0.3 μm to 1.1 μm, or another wavelength band. Various modifications can be made according to the characteristics of the sensitivity wavelength range of the light receiver 44 or the like.

As shown in FIG. 1A etc., the biological information detection apparatus 10 in which the casing having a radius of rN around the light receiver 44 is provided as the light-blocking member is considered. In this case, sunlight enters all points at which distances from the light receiver 44 are larger than rN. Note that, actually, the size of the body of the user is finite and, as shown in FIG. 3, as the propagation distance within the living body is larger, the light power exponentially decreases. Thus, for a point that the distance from the light receiver 44 is larger that rN, the need to consider the point at infinity from the light receiver 44 is lower and, here, a range rN<r<30 mm is considered. Further, as described above, only the wavelength band of sunlight from 0.4 μm to 1.0 μm is considered.

FIG. 6 shows a specific calculation example. FIG. 6 shows a region cocentrically sectioned around the light receiver 44. B5 in FIG. 6 is a region representing a circle with a radius of 5 mm around the light receiver 44.

At a point at distance r from the light receiver 44, i.e., at a point on the circle of radius r around the light receiver 44, light from 0.4 μm to 0.5 μm enters with intensity of 0.130 mW/mm² (FIG. 5), attenuated at a rate corresponding to R_{0.4-0.5}(r) in FIG. 3, and enters the light receiver 44 with power of R_{0.4-0.5}(r)×0.130 mW/mm². Similarly, in consideration of the wavelength band to 1.0 μm, the light entering the point at the distance r from the light receiver 44 is attenuated to light power pn expressed by the following expression (6) and enters the light receiver 44.

pn=R_{0.4-0.5}(r)×0.130+R_{0.5-0.6}(r)×0.155+R_{0.6-0.7}(r)×0.145+R_{0.7-0.8}(r)×0.126+R_{0.8-0.9}(r)×0.102+R_{0.9-1.0}(r)×0.083 (mW/mm²) (6)

In consideration of the expression (6) expressing light power entering one point at distance r from the light receiver 44 and passing through the living body, the expression (6) is integrated with respect to a whole range that satisfies rN<r<30 mm in FIG. 6 (in a polar coordinate system, θ is from 0 to 2π), and thereby, power PN of disturbance light for the casing size rN may be obtained.

Regarding actual calculation, coaxial regions may be set as shown in FIG. 6 and calculation may be performed with respect to each region. For example, the B5 region has an area of (6²−5²)π (mm²). Within B5, points at distances of 5 mm to light receiver 44, points at distances of 6 mm, and points at intermediate distances are mixed, and the degree of attenuation of light power is represented by an average value of the degree of attenuation at 5 mm and the degree of attenuation at 6 mm. For example, the degree of attenuation with respect to light from 0.4 μm to 0.5 μm entering points within the region B5 may be considered as R′_{0.4-0.5} of the following expression (7).

R′_{0.4-0.5}(B5)={R_{0.4-0.5}(5)+R_{0.4-0.5}(6)}/2 (7)

Similarly, supposing that the degrees of attenuation of light power entering the region B5 is represented by average values R′ of the degrees of attenuation at 5 mm and the degrees of attenuation at 6 mm with respect to the other wavelength bands, light power pn(B5) of light entering the region B5, passing through the living body, and entering the light receiver 44 is obtained by the following expression (8).

pn(B5)={R′_{0.4-0.5}(B5)×0.130+R′_{0.5-0.6}(B5)×0.155+R′_{0.6-0.7}(B5)×0.145+R′_{0.7-0.8}(B5)×0.126+R′_{0.8-0.9}(B5)×0.102+R′_{0.9-1.0}(B5)×0.083}×(6²−5²)π (8)

Similarly, regarding a region B6 at a distance from 6 mm to 7 mm from the light receiver 44, a region B7 at a distance from 7 mm to 8 mm, etc., light power pn(Bi) entering a region Bi at a distance from i mm to i+1 mm from the light receiver 44, passing through the living body, and entering the light receiver 44 may be obtained. Here, as described above, the range of distance from the light receiver 44 equal to or less than 30 mm is considered, and the light power to pn(B29) may be obtained.

In this manner, with respect to each concentric region, light power pn of sunlight entering the region, passing through the living body, and entering the light receiver 44 may be obtained. Here, as shown in FIG. 1A etc., the case where a casing having a circular plate shape of radius 5 (mm) is used as the casing of the biological information detection apparatus 10 and a light-blocking area with a radius of 5 mm is set by the casing is considered. In this case, entrance of sunlight into the living body in a range within 5 mm from the light receiver 44 is suppressed and, as a region that sunlight enters, a collective region of B5, B6, . . . , B29 may be considered. Accordingly, PN is a sum of pn(B5) to pn(B29).

This is generalized, and the case where a casing having a circular plate shape of radius rN (mm) is used as the casing of the biological information detection apparatus 10 and a light-blocking area with a radius of rN mm is set by the casing is considered. In this case, as a region that sunlight enters, a collective region of BrN, BrN+1, . . . , B29 may be considered. Accordingly, PN(rN) can be obtained by the following expression (9).

$$PN ( rN ) = {\sum }_{i = rN}^{29} pn ( Bi ) \qquad ( 9 )$$

As shown in FIG. 3, the numerical values of R_{0.4-0.5}(r) are known. Therefore, specific values are substituted into the expression (9), and thereby, a numerical value relationship between PN and rN may be obtained as shown in FIG. 7. Note that, as above, the processing on the wavelength band from 0.4 μm to 1.0 μm is performed, however, as described above, the degree of attenuation of the light having the shorter wavelength with respect to the propagation distance r is larger and its contribution to PN is smaller. Therefore, for example, a modification that the processing on the wavelength band of the relatively short wavelength from 0.4 μm to 0.6 μm is omitted and the above described processing is performed on the wavelength band from 0.6 μm to 1.0 μm can be made. Further, here, the value equal to or more than 5 mm is assumed as rN and the regions B5 to B29 are considered, however, a region nearer the light receiver 44, e.g. a region B4 at a distance from 4 mm to 5 mm from the light receiver 44 may be considered, and, in this case, PN(rN) for rN<5 mm can be obtained.

If disturbance light is not attenuated at the attenuation rate α and the above described condition equal to or less than 4.71×10⁻⁶ mW/mm² is satisfied by the size rN of the biological information detection apparatus 10, e.g. rN=17 mm is required from FIG. 7. PN is 3.36×10⁻⁶ mW/mm² for rN=17 mm from FIG. 7, and satisfies the condition equal to or less than 4.71×10⁻⁶ mW/mm².

However, in this case, rN becomes too large and, for example, supposing that the biological information detection apparatus 10 is a wristwatch-type apparatus and the rN is a radius of a main body part having a circular plate shape, the diameter of the main body part is 34 mm. As known from imagination of a face of a watch, the size with the diameter of 34 mm is very large and not preferable because wearing is bothersome.

Accordingly, in the embodiment, light power PN of disturbance light passing through the living body and entering the light receiver 44 is attenuated to α times (<1). In this manner, light power corresponding to noise may be sufficiently made smaller by setting α×PN≦4.71×10⁻⁶ mW/mm², not the PN itself obtained from FIG. 7 to 4.71×10⁻⁶ mW/mm². Namely, rN can be made smaller and the biological information detection apparatus 10 can be downsized.

For example, as described above, rN≦10 mm, i.e., the size with a diameter of 20 mm is a desirable value in consideration of attachment to an arm or the like. From FIG. 7, PN(10)=1.40×10⁻⁴ mW/mm², and, supposing that α=1/30, α×PN(10)=1.40×10⁻⁴×1/30=4.67×10⁻⁶ mW/mm²≦4.71×10⁻⁶, and both the expression (1) and the expression (2) can be satisfied. Further, the condition can be satisfied by further increasing the degree of attenuation, in other words, further decreasing α less than 1/30.

That is, the attenuation rate α of disturbance light is set to satisfy rN≦10 mm and α≦1/30, and thereby, the biological information detection apparatus 10 that can appropriately detect biological information (pulse wave information) can be realized.

In this regard, the attenuation rate α of disturbance light is set so that α may be smaller in the range of α≦1/30 as rN is smaller in the range of rN≦10. Namely, the influence by disturbance light is larger as rN is smaller, and the influence by disturbance light can be appropriately suppressed by increasing the degree of attenuation (decreasing α).

For example, the attenuation rate α of disturbance light may be set to satisfy rN≦8 mm and α≦1/100. From FIG. 7, PN(8)=4.58×10⁻⁴ mW/mm², and, supposing that α=1/100, α×PN(8)=4.58×10⁻⁶ mW/mm²≦4.71×10⁻⁶, and both the expression (1) and the expression (2) can be satisfied.

Similarly, the attenuation rate α of disturbance light may be set to satisfy rN≦5 mm and α≦1/1000. From FIG. 7, PN(5)=3.42×10⁻³ mW/mm², and, supposing that α=1/1000, α×PN(5)=3.42×10⁻⁶ mW/mm²≦4.71×10⁻⁶, and both the expression (1) and the expression (2) can be satisfied.

### 2.2 Specific Configuration for Realization of Attenuation Rate α

As described above, in the embodiment, light power PN of disturbance light from outside of the end portion of the contact part 50 passing through the living body and entering the light receiver 44 is attenuated to α times. In this regard, to realize downsizing of rN≦10, it is necessary to set α to a very small value from 1/30 to 1/1000 as described above. There is a typical product of related art provided with an optical filter or the like, however, the attenuation rate thereof remains about a fraction (specifically, more than about 1/10), and it is considered that a high-performance optical filter is harder to mount.

However, the applicant can mount a high-performance optical filter having an attenuation rate from 1/30 to 1/1000. FIG. 8 shows a specific example of the attenuation rate. As described above, the wavelength band of signal light is transmitted and light power of lights of the other wavelength bands are attenuated to α times. Here, the wavelength band of signal light is from 0.5 μm to 0.6 μm, and the transmittance of the band may be set to 100% and the attenuation rate of the other wavelength bands may be set to α. Further, as described above, if the wavelength bands equal to or more than 1.0 μm is negligible, it is not necessary to set the attenuation rates of the wavelength bands to α. For example, the transmittance may be set to 100%.

That is, the biological information detection apparatus 10 according to the embodiment further includes an optical filter provided in the light receiver 44 and limiting light entering the light receiver 44, and the attenuation rate α may be set by the optical filter. Thereby, the attenuation rate α may be realized by providing the optical filter.

In this regard, the optical filter may include a multilayer filter (dielectric multilayer film 111). The above described very small α can be realized using the multilayer filter.

FIG. 9 shows a configuration example (sectional view) of one light receiving sensor 140 cut out from a semiconductor wafer by dicing. As shown in FIG. 9, each sensor of a plurality of light receiving sensors 140 cut out from the semiconductor wafer includes a semiconductor substrate 101, a dielectric multilayer film 111, and a photodetection region 121. The photodetection region 121 here corresponds to the light receiver 44, e.g., a photodiode (PD), and is specifically realized by a P-N junction diode or the like. The sensitivity wavelength range of the photodetection region 121 changes depending on doping concentration when the P-N junction is formed or the like, e.g. a wavelength band from about 300 to 1100 nm (0.3 to 1.1 μm) or from about 400 to 1000 nm (0.4 to 1.0 μm).

As shown in FIG. 9, the dielectric multilayer film 111 (multilayer optical filter) is a film in which a first refractive index layer having a first refractive index and a second refractive index layer having a second refractive index lower than the first refractive index are stacked. As shown in FIG. 9, the first refractive index layer (hereinafter, referred to as “high refractive index layer”) may be a layer of titanium oxide (specifically, titanium dioxide TiO2) and the second refractive index layer (hereinafter, referred to as “low refractive index layer”) may be a layer of silicon oxide (specifically, silicon dioxide SiO2).

In consideration of optical processing, in a given path (optical path), a change point of refractive index may be provided. In this case, a surface on which the high refractive index layer and the low refractive index layer are in contact is the change point of refractive index. That is, for stacking the high refractive index layers and the low refractive index layers, the high refractive index layers and the low refractive index layers may be alternately stacked as shown in FIG. 9. Note that the high refractive index layers and the low refractive index layers are alternately stacked along a direction (nearly) perpendicular to the semiconductor substrate 101. In FIG. 9 etc., both end surfaces in the stacking direction are the high refractive index layers, and the number of stacked layers is an odd-number (e.g. 61 or the like), but not limited to that.

Further, the dielectric multilayer film 111 is a filter serving as a bandpass filter. It is known that the above described high refractive index layers using TiO2 and the low refractive index layers using SiO2 are stacked, and thereby, light in a predetermined wavelength band can be stopped. More specifically, about twenty of the high refractive index layers and the low refractive index layers in total are stacked, and thereby, a wavelength band of about 200 nm can be stopped.

In the embodiment, the wavelength bands around the passband are blocked by the dielectric multilayer film 111, and thereby, a bandpass filter having a desired passband is realized. For example, when the passband is from 500 to 600 nm, stop wavelength bands may be set from 300 to 500 nm and from 600 to 1100 nm. This corresponds to a property of cutting ultraviolet light and blue light (from 300 to 500 nm), red light (from 600 to 700 nm), and near-infrared light (from 700 to 1100 nm). In this regard, not all layers of the dielectric multilayer film 111 have properties of stopping lights from 300 to 500 nm and from 600 to 1100 nm, and the dielectric multilayer film 111 may be divided and considered in several groups.

Specifically, the dielectric multilayer film 111 is an optical filter having a first group of refractive index layers and a second group of refractive index layers, in which a first frequency band is attenuated in the first group of refractive index layers, a second frequency band is attenuated in the second group of refractive index layers, and a third frequency band between the first frequency band and the second frequency band serves as a passband.

For example, twenty layers of the dielectric multilayer film 111 are used for stopping lights from 300 to 500 nm. In this case, the twenty layers are the first group of refractive index layers and the band from 300 to 500 nm is the first frequency band. Further, forty layers of the dielectric multilayer film 111 are used for stopping lights from 600 to 1100 nm. In this case, the forty layers are the second group of refractive index layers and the band from 600 to 1100 nm is the second frequency band. The third frequency band in this case is from 500 to 600 nm.

As described above, the first and second groups of refractive index layers are not formed by stacking twenty or forty of either high refractive index layers or low refractive index layers, but respectively include high refractive index layers and low refractive index layers (in a narrow sense, alternately stacked). Or, the second group of refractive index layers may be further divided and considered. For example, lights from 600 to 800 nm may be stopped (attenuated) using twenty layers of the second group of refractive index layers and lights from 800 to 1100 nm may be stopped using the other twenty layers of the second group of refractive index layers.

In this manner, a bandpass filter with very high accuracy can be realized. A color filter provided in an image sensor (imaging sensor) of a digital camera or the like is also a bandpass filter having a passband in a specific wavelength range of visible light, however, its accuracy is very low. This is because, in the image sensor, received signals are converted into images and presented to the user. Accordingly, even when light in a different wavelength band from the desired wavelength band is received, a resulting output image does not largely change and, particularly, it is hard for human eyes to found a problem. It is intended that the light receiving sensor according to the embodiment includes a bandpass filter with higher accuracy and, in order to realize e.g. the above described α, the transmittance of the signal in the stopband is suppressed to from 1/100 to 1/10000 or less. For the purpose, as described above, the dielectric multilayer film in which many layers with different refractive indexes are stacked may be used.

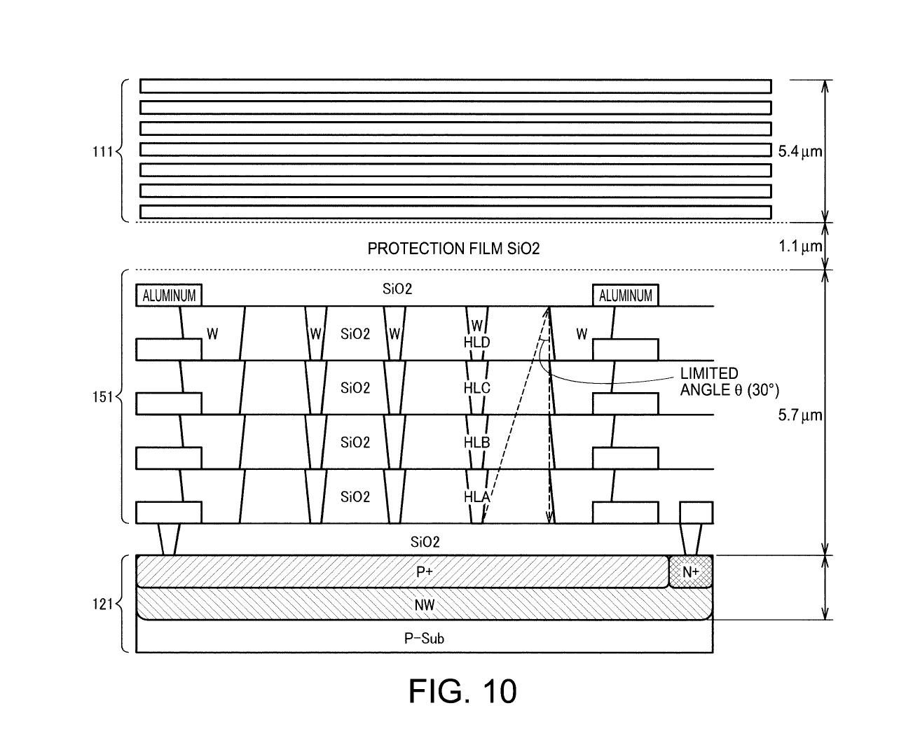

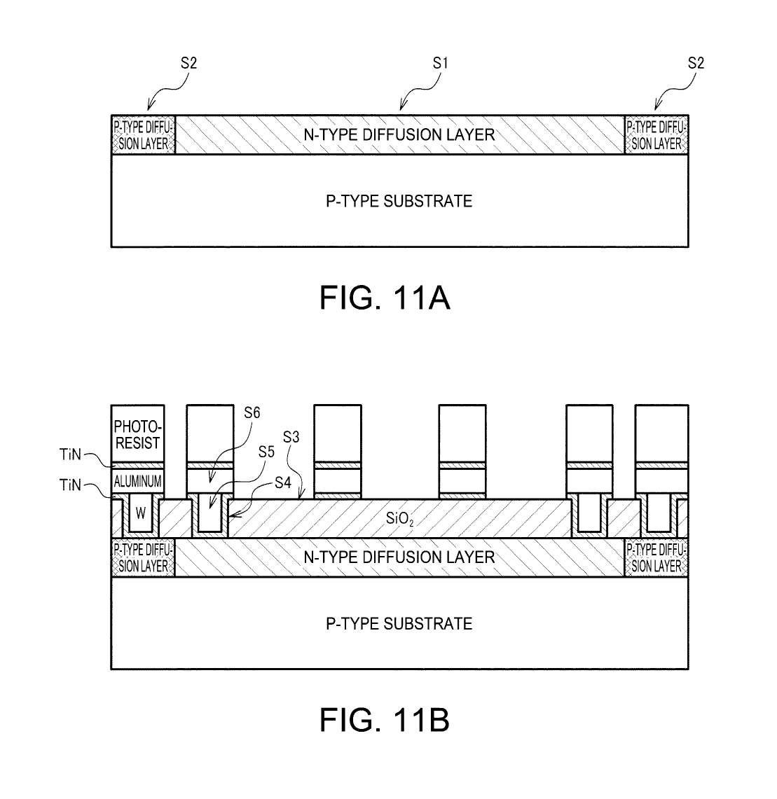

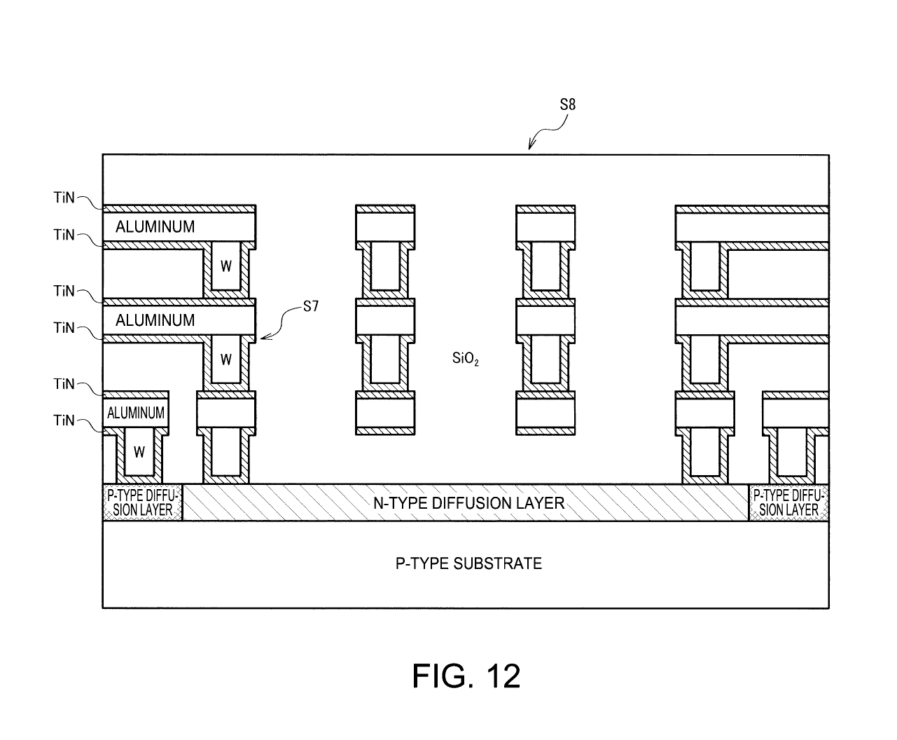

Further, for realization of the attenuation rate α, another filter than the multilayer filter may be provided. For example, as shown in FIG. 10, an angle-limiting filter 151 may be provided between the dielectric multilayer film 111 and the photodetection region 121, and α may be realized by both the multilayer filter and the angle-limiting filter 151. Alternatively, the multilayer filter is not provided, and α may be realized by the angle-limiting filter 151. FIGS. 11A to 12 show an example of a process of forming the angle-limiting filter 151.

First, as shown by S1 in FIG. 11A, an N-type diffusion layer (an impurity region of a photodiode) is formed on a P-type substrate at steps of photolithography, ion implantation, and photoresist stripping. As shown by S2, P-type diffusion layers are formed on the P-type substrate at steps of photolithography, ion implantation, photoresist stripping, and heat treatment. The N-type diffusion layer serves as a cathode and the P-type diffusion layer (P-type substrate) serves as an anode of the photodiode.

Then, as shown by S3 in FIG. 11B, insulating films are formed at step of planarization by deposition of SiO2 and polishing (e.g. CMP (Chemical Mechanical Polishing)). As shown by S4, contact holes are formed at steps of photolithography, anisotropic dry etching of SiO2, and photoresist stripping. As shown by S5, embedding of contact holes is performed at steps of sputtering of TiN (titanium nitride), deposition of W (tungsten), and etchback of W. As shown by S6, a first level of aluminum interconnections is formed at steps of sputtering of aluminum, sputtering of TiN, photolithography, anisotropic dry etching of aluminum and TiN, and photoresist stripping.

Then, as shown by S7 in FIG. 12, via contacts and a second level of aluminum interconnections are formed at the same steps as S3 to S6. Subsequently, the step S7 is repeated at a required number of times. FIG. 12 shows the case where a third level of aluminum interconnections is further formed. Furthermore, as shown by S8, insulating films are formed at steps of deposition of SiO2 and planarization by CMP. Through the above described interconnection forming process, aluminum interconnections and tungsten plugs forming the angle-limiting filter are stacked. Note that the angle-limiting filter 151 in FIG. 10 is an example in which aluminum interconnections up to the fifth level are formed. Various modifications can be made to the configuration of the angle-limiting filter 151 as long as a condition of an angle θ, which will be described later, is satisfied.

The tungsten plugs shown by W in FIG. 10 are provided, and thereby, the angle θ is limited to 30 degrees. Accordingly, as shown in FIG. 10, of lights to enter the photodetection region 121, lights at incident angles (angles with respect to a direction perpendicular to the surface of the photodetection region) less than 30 degrees may reach the photodetection region 121, but lights at incident angles equal to or more than 30 degrees may not reach the photodetection region 121 (blocked by the tungsten plugs before reaching).

Further, as in the above described example of receiving lights from 0.3 to 1.1 μm, it is known that the light receiver 44 (photodiode) itself has sensitivity depending on wavelength. That is, the attenuation rate α is not limited to that realized by at least one of the multilayer filter and the angle-limiting filter 151, but may be realized by at least one of the multilayer filter and the angle-limiting filter 151 and the property of the light receiver 44 itself.

### 3. Configuration Example of Biological Information Detection Apparatus

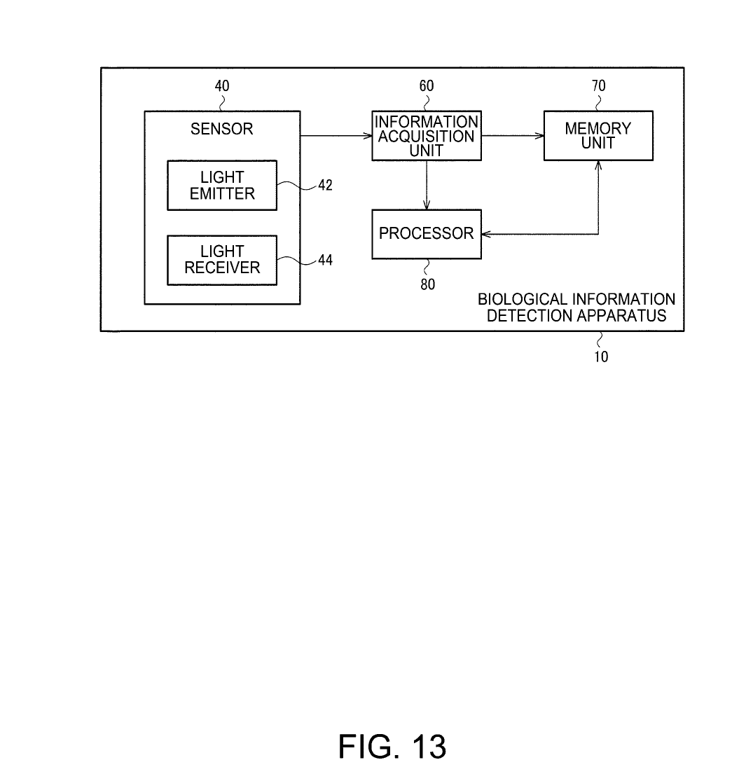

Next, a configuration example of the biological information detection apparatus 10 according to the embodiment is explained. As shown in FIG. 13, the biological information detection apparatus 10 according to the embodiment includes an information acquisition unit 60, a memory unit 70, and a processor 80. Note that the biological information detection apparatus 10 is not limited to the configuration in FIG. 13, but various modifications of addition of another element or the like can be made.

The information acquisition unit 60 acquires sensor information (pulse wave sensor information) from the sensor 40 (pulse wave sensor) including the light emitter 42 and the light receiver 44.

The memory unit 70 serves as a work area for the processor 80 etc., and its function may be realized by a memory such as a RAM, an HDD (hard disc drive), or the like. The memory unit 70 stores the pulse wave sensor information acquired by the information acquisition unit 60. The memory unit 70 may store processing results in the processor 80.

The processor 80 performs detection processing of biological information based on the sensor information from the sensor 40. Specifically, the sensor information is the pulse wave sensor information and the biological information is the pulse wave information. In this manner, the influence of disturbance light can be appropriately suppressed and the biological information (in a narrow sense, pulse wave information) can be detected with high accuracy in the biological information detection apparatus 10.

FIGS. 14A to 15 show examples of appearance diagrams of the biological information detection apparatus 10 that collects biological information (in the narrow sense, pulse wave information). The biological information detection apparatus 10 of the embodiment has a band part 11, a case part 30, and the sensor 40. The case part 30 is attached to the band part 11. The sensor 40 is provided in the case part 30.

The band part 11 is wrapped around the wrist of the user for attaching the biological information detection apparatus 10. The band part 11 has band holes 12 and a buckle part 14. The buckle part 14 has a band insertion portion 15 and a projecting portion 16. The user inserts one end side of the band part 11 into the band insertion portion 15 of the buckle part 14, inserts the projecting portion 16 of the buckle part 14 into the band hole 12 of the band part 11, and thereby, attaches the biological information detection apparatus 10 to the wrist. Note that the band part 11 may have a clasp in place of the buckle part 14.

The case part 30 corresponds to the main body part of the biological information detection apparatus 10. Inside of the case part 30, various component parts of the biological information detection apparatus 10 including the sensor 40, a circuit board (not shown), etc. are provided. In other words, the case part 30 is a casing that houses these component parts.

A light emitting window portion 32 is provided in the case part 30. The light emitting window portion 32 is formed using a light-transmissive member. Further, a light emitter as an interface mounted on a flexible board is provided in the case part 30, and light from the light emitter is output to the outside of the case part 30 via the light emitting window portion 32.

The biological information detection apparatus 10 is attached to the wrist of the user, and the pulse wave information (in a broad sense, biological information) is measured in the attached state. Specifically, the sensor 40 includes the photoelectric sensor having the light emitter 42 and the light receiver 44, and measures the pulse wave information using the photoelectric sensor. The measurement of the pulse wave information using the photoelectric sensor is a widely known technique, and the detailed explanation is omitted. Further, the attachment part of the biological information detection apparatus 10 may be an ankle, a finger, an upper arm, or the like.

The biological information detection apparatus 10 is not limited to the band-type (wristwatch-type) apparatus in FIGS. 14A to 15, but various apparatuses including a band-type apparatus worn on the chest, an apparatus adhesively attached to a neck part or the like, and a spectacle-type (face mounted) apparatus can be employed. Further, the mounted sensor is not limited to the photoelectric sensor, but various sensors including an ultrasonic sensor, a body motion sensor (e.g. acceleration sensor), and a position sensor such as a GPS receiver can be used.

As shown in FIGS. 14A to 15 etc., the biological information detection apparatus 10 may include a casing (e.g. the case part 30) in which the sensor 40 is provided. In this case, the contact part 50 in contact with the living body is a casing surface on which the casing comes into contact with the user when the casing is attached to the subject (user). In this manner, the contact part 50 may be realized by bringing a given surface of the casing into contact with the living body, and the light-blocking area can be realized by the casing. In this case, the distance rN to the end portion of the contact part 50 corresponds to the casing size, and the casing size (in a broad sense, the size of the biological information detection apparatus 10) can be reduced by reducing rN.

Obviously, the casing of the biological information detection apparatus 10 is the circular plate shape, and is not limited to the casing with the circular casing surface as the contact surface. In other words, the shape is not limited to the shape in which the distance from the light receiver 44 to the end portion of the contact part 50 is a constant value (rN) at points on all end portions, but generally a more complex shape. In this case, various techniques of setting “the distance rN from the light receiver 44 to the end portion of the contact part 50” are conceivable. For example, if importance is placed on the detection accuracy of biological information, it is safer to estimate the light-blocking area to be smaller than the real one. That is, the minimum value of the distances from the light receiver 44 to the end portion of the contact part 50 may be set to rN. Note that, here, PM is PS/1000, however, actually from about 1/800 to 1/300 including a margin. Namely, a modification in which the maximum value or an average of the distances from the light receiver 44 to the end portion of the contact part 50 is set to rN can be made.

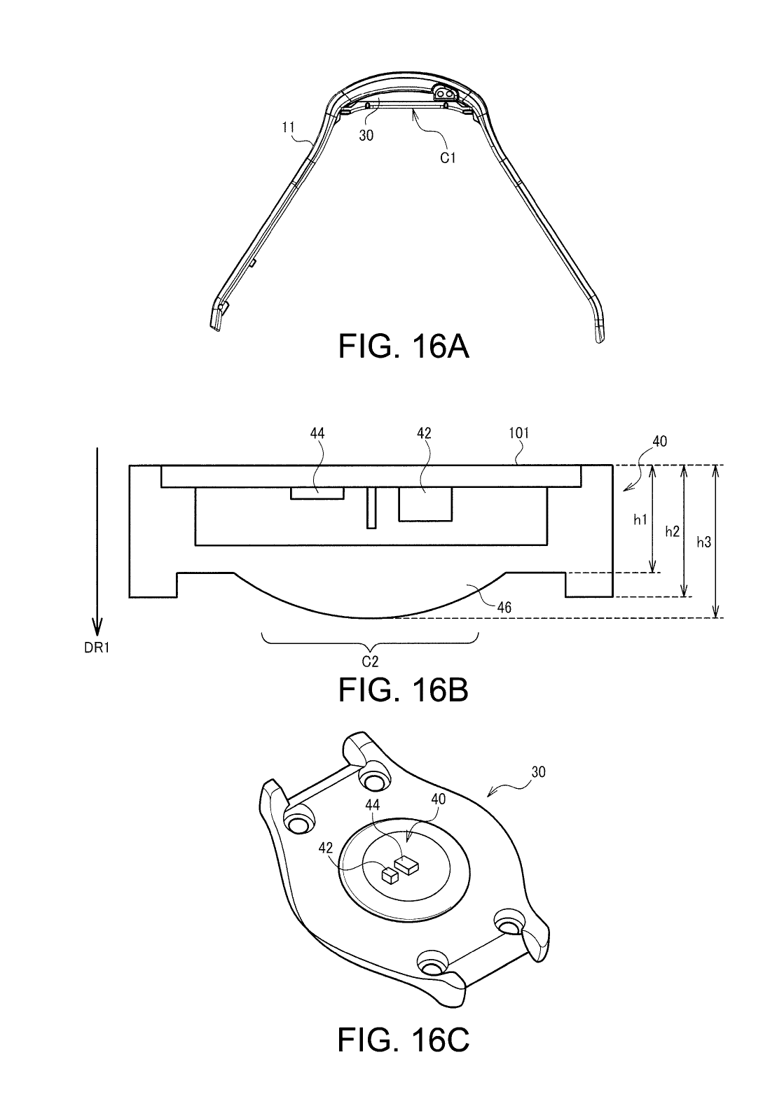

Further, the contact part 50 according to the embodiment can be considered in another point of view. For example, the biological information detection apparatus 10 may include a casing (e.g. the case part 30) in which the sensor 40 is provided, and the contact part 50 may be a casing surface facing a subject when the biological information detection apparatus 10 is attached to the subject. As one example, the case part 30 includes the back cover part as described above, and the contact part 50 is a surface of the back cover part facing the subject in the attached state. For example, when the appearance diagram of the biological information detection apparatus 10 is FIG. 16A, the contact part 50 is realized by a surface shown by C1 of the case part 30.

Not all of the surfaces facing the subject in the attached state are required to correspond to the contact part 50. For example, the contact part 50 may be a projecting region projecting toward the subject of the surfaces of the casing facing the subject in the state in which the biological information detection apparatus 10 is attached to the subject.

In the detection of biological information (pulse wave information), it is necessary to apply an appropriate pressing force to a part of the subject to be measured. This is because, in the pulse wave information, an arterial signal is a detection object and a venous signal is a noise factor, and pressing forces by which the signals are lost (the blood flows in the vessels are sufficiently weak) are different between an artery and a vein. Specifically, supposing that the pressing force corresponding to the vein loss point at which the venous signal is sufficiently small is P1 and the pressing force corresponding to the artery loss point at which the arterial signal is sufficiently small is P2, P1<P2. Accordingly, a pressing force P that satisfies P1<P<P2 is applied, and thereby, the influence by the venous signal may be suppressed while the arterial signal is detected, and pulse wave information detection with high accuracy can be performed.

As described above, in the detection of biological information, the pressing force on the subject is important and, in order to efficiently realize the pressing force, a projecting region may be provided on the surface on the subject side of the case part 30. In the biological information detection apparatus 10 shown in FIG. 15, a part of the case part 30 in which the sensor 40 is provided corresponds to the projecting region. Specifically, as shown in FIG. 16B, a light-transmissive member 46 may be provided on the surface on the subject side of the back cover, and the light-transmissive member 46 may have a structure projecting toward the subject side compared to the other parts of the back cover in a first direction DR1 (the first direction DR1 is a direction from the biological information detection apparatus 10 toward the subject). According to the configuration, when the biological information detection apparatus 10 is attached to the subject, load by a load mechanism (e.g. the band part 11 in FIG. 14A) is easily concentrated on the projecting region, and thereby, the desired pressing force can be efficiently realized.

Or, a given reference surface may be set and whether or not a region projects may be determined based on its height relative to the reference surface. As one example, a surface of the semiconductor substrate 101 on which the light emitter 42 and the light receiver 44 are provided or a surface parallel to the semiconductor substrate 101 is set as the reference surface. In the example of FIG. 16B, of the semiconductor substrate 101, a rear surface of the surface provided with the light emitter 42 etc. is set as the reference surface and a region having a height h relative to the reference surface is larger than the surrounding portion is determined as the projecting region. Here, a distance in a direction crossing (in a narrow sense, orthogonal to) the reference surface may be used as the height relative to the reference surface, and, in the example of FIG. 16B, the above described first direction DR1 is the direction crossing the reference surface. In FIG. 16B, the light-transmissive member 46 includes a surface having the height of h1, a surface having a height of h2, and a curved surface having a height changing in a range from h1 to h3 relative to the reference surface, and e.g. a region of the curved surface having the height equal to or more than h2 may be considered as the projecting region.

In the case of FIG. 16B, the region shown by C2 is the projecting region and the projecting region serves as the contact part 50 of the embodiment. In this manner, an appropriate pressing force on the living body may be realized by the projecting region and the projecting region is used as the contact part 50, and thereby, settings of parameters that appropriately reduce the influence by outside light can be made. Note that the projecting region is not limited to that formed by the light-transmissive member 46, but may have a structure as shown in FIG. 16C. FIG. 16C is a perspective view of the case part 30 of the biological information detection apparatus 10 observed from the subject side at attachment. In FIG. 16C, the light emitter 42 and the light receiver 44 themselves project toward the subject side and the light emitter 42 and the light receiver 44 serve as projecting regions (and the contact part 50). Or, another modification than those in FIGS. 16B and 16C can be made to the projecting region.

Further, as described above, the biological information detection apparatus 10 according to the embodiment is not limited to the apparatus attached to an arm, but may be a clip-shaped or ring-shaped apparatus attached to a finger or an apparatus worn on the face in a spectacle shape or the like.

Or, the biological information detection apparatus 10 according to the embodiment is not limited to the apparatus including the distinct casing, but may be a seal-type apparatus. For example, an apparatus like an adhesive bandage that is widely used for protection of a wound or the like may be employed. The light emitter 42 and the light receiver 44 according to the embodiment may be provided in a position in which a gauze patch to be in contact with the wound is provided in the case of the adhesive bandage. In this case, the biological information detection apparatus 10 does not include the casing formed of a resin or the like, but realizes the light-blocking area by the tape part having adhesion. It is desirable that the seal-type biological information detection apparatus 10 is readily available and downsizing is strongly requested in consideration of a risk of skin irritation or the like. Namely, the apparatus has very high affinity for the technique of the embodiment that enables appropriate downsizing.

Further, the technique of the embodiment can be applied to an electronic apparatus including the above described biological information detection apparatus 10.

### 4. Modified Examples

As above, the explanation is made assuming that PS takes the value of the expression (5). As described above, in the present circumstances, products (in a narrow sense, biological information detection apparatuses) using various optical sensors are known, and the signal levels (light power corresponding to signals) nearly at the same level in many products are considered.

However, as described above, the value of PS can be changed by various settings. For example, power of light radiated from the light emitter 42 is increased or the directivity of light is improved by providing a lens or the like, and thereby, light power entering the light receiver 44 can be increased. Or, even when lights having the same power enter, the light sensitivity of the light receiver 44 is increased at least in the wavelength band of the signal light LS, and thereby, the signal component in the output of the light receiver 44 can be increased. In this manner, the properties of the light emitter 42 and the light receiver 44 are changed, and thereby, PS can be increased. In this case, specifically, the value “1 mW” in the first line of the expression (5) increases.

Or, as shown in FIG. 3, as the propagation distance within the living body is larger, the degree of attenuation of light power is larger. Therefore, the distance between the light emitter 42 and the light receiver 44 is made shorter, and thereby, PS can be increased.

That is, in the above described example, PS taking a nearly constant value is considered, however, actually, PS is a variable depending on the property L of the light emitter 42, the property P of the light receiver 44, and the distance rS between the light emitter 42 and the light receiver 44. In other words, PS is a function PS(L,P,rS) of L, P, and rS. Therefore, not only the parameter settings in consideration of the above described relationship between rN and α, but also settings of the parameters L, P, rS that determine PS in consideration of relationships may be made.

In this case, the expression (2) is expressed by the following expression (10).

α×PN(rN)≦PS(L,P,rS)/1000 (10)

That is, in a modified example of the embodiment, in the biological information detection apparatus 10, at least five parameters α, rN, L, P, rS may be set to satisfy the relationships of the expression (1) and the expression (10). For example, supposing that α, L, P are given values, rN and rS may be set to satisfy α×PN(rN)≦PS(rS)/1000. As rN is made larger, the S/N ratio can be further improved, and, as rS is made smaller, the S/N ratio can be further improved.

In this case, rN and rS relate to the dimensions of the biological information detection apparatus 10, and both detection accuracy of biological information and downsizing can be compatibly realized depending on the size, shape, part arrangement of the biological information detection apparatus 10. For example, when the minimum value of rS (the minimum distance at which the light emitter 42 and the light receiver 44 are made closest to each other) is determined from a problem of arrangement accuracy of parts or the like, the minimum value of rN, i.e., a relationship with which the limit of downsizing of the biological information detection apparatus 10 is determined is obtained. Conversely, if a condition to desire rN equal to or less than a predetermined value is determined as a request of downsizing, the value of rS for appropriate detection of biological information under the condition, i.e., an arrangement condition in which the light emitter 42 and the light receiver 44 should be made closer to each other is determined.

One example of the parameter L is light emission intensity of the light emitter 42 (LED power). In this case, as L is made larger, the S/N ratio is further improved. Accordingly, the conditions on rN, α, rS, etc. can be relaxed by increasing L. For example, further downsizing can be realized by decreasing rN, a performance request for the optical filter can be relaxed by increasing α, or a restriction on the part arrangement can be relaxed by increasing rS.

In addition, in the modified example, various parameters can be flexibly set under the condition that satisfies the expressions (1) and (10). Note that, as a specific example of the parameter P, sensitivity of the light receiver 44 is considered and, more specifically, may be a parameter that changes sensitivity in a given wavelength band with the sensitivity wavelength range as it is or a parameter that changes the sensitivity wavelength range itself of the light receiver 44. Note that, regarding the parameter P, if P is changed, not only PS but also PN may change. In other words, PN may be a function PN(fN,P) of rN and P. Namely, it is preferable to set the parameter P in careful consideration of settings that improve the S/N ratio.

The embodiment is explained in detail as described above, a person skilled in the art could readily understand that many modifications can be made without substantially departing from the new matter and effects of the invention. Therefore, these modified examples fall within the scope of the invention. For example, in the specification and the drawings, terms described with different terms in broader senses or the same meanings at least once may be replaced by the different terms in any part of the specification and the drawings. Further, the configurations and operations of the biological information detection apparatus, the electronic apparatus, etc. are not limited to those explained in the embodiment, but various modifications can be made.

What is claimed is:

1. A biological information detection apparatus comprising:
a sensor having a light emitter that radiates light to a subject and a light receiver that receives light from the subject; and
a contact part that is in contact with the subject,
wherein, supposing that light power from the light emitter passing through a living body as the subject and entering the light receiver is PS and a distance from the light receiver to an end portion of the contact part is rN, and light power of disturbance light from outside of the end portion passing through the living body and entering the light receiver is PN(rN) as a function of rN, an attenuation rate α of the disturbance light satisfies
rN≦10 mm and α≦PS/{PN(rN)×1000}.

2. The biological information detection apparatus according to claim 1, wherein the attenuation rate α satisfies rN≦10 mm and α≦1/30.

3. The biological information detection apparatus according to claim 1, wherein the attenuation rate α satisfies rN≦8 mm and α≦1/100.

4. The biological information detection apparatus according to claim 1, wherein the attenuation rate α satisfies rN≦5 mm and α≦1/1000.

5. The biological information detection apparatus according to claim 1, further comprising an optical filter provided in the light receiver and limiting light entering the light receiver.

6. The biological information detection apparatus according to claim 5, wherein the optical filter includes a multilayer filter.

7. The biological information detection apparatus according to claim 1, further comprising a casing in which the sensor is provided,
wherein the contact part is a casing surface on which the casing is in contact with the subject when the casing is attached to the subject.

8. The biological information detection apparatus according to claim 1, further comprising a casing in which the sensor is provided,
wherein the contact part is a surface of the casing facing the subject when the casing is attached to the subject.

9. The biological information detection apparatus according to claim 1, further comprising a casing in which the sensor is provided,
wherein the contact part is a projecting region projecting toward the subject of a surface of the casing facing the subject when the casing is attached to the subject.

10. The biological information detection apparatus according to claim 1, further comprising a processor that performs detection processing of biological information based on sensor information from the sensor.

11. The biological information detection apparatus according to claim 10, wherein the biological information is pulse wave information.

12. The biological information detection apparatus according to claim 11, wherein, supposing that light power from the light emitter passing through the living body, entering the light receiver, and corresponding to the pulse wave information is PM,
PM≧PS/1000.

13. An electronic apparatus including the biological information detection apparatus according to claim 1.

14. An electronic apparatus including the biological information detection apparatus according to claim 2.

15. An electronic apparatus including the biological information detection apparatus according to claim 3.

16. An electronic apparatus including the biological information detection apparatus according to claim 4.

17. An electronic apparatus including the biological information detection apparatus according to claim 5.

18. An electronic apparatus including the biological information detection apparatus according to claim 6.

19. An electronic apparatus including the biological information detection apparatus according to claim 7.

20. An electronic apparatus including the biological information detection apparatus according to claim 8.
